(19) United States
(12) Patent Application Publication
(10) Pub. No.: US 2024/0423806 A1
(43) Pub. Date: Dec. 26, 2024

(54) ACTUATOR FOR HIP REPLACEMENT AND SURGICAL SYSTEM

(71) Applicant: BEIJING HURWA MEDICAL TECHNOLOGY CO., LTD., Beijing (CN)

(72) Inventors: Zhao ZHANG, Beijing (CN); Xiaofeng ZHANG, Beijing (CN); Wei LI, Beijing (CN); Chao WANG, Beijing (CN); Jinbang GUO, Beijing (CN); Chun ZHANG, Beijing (CN)

(73) Assignee: BEIJING HURWA MEDICAL TECHNOLOGY CO., LTD., Beijing (CN)

(21) Appl. No.: 18/824,510

(22) Filed: Sep. 4, 2024

Related U.S. Application Data
(63) Continuation of application No. PCT/CN2023/103989, filed on Jun. 29, 2023.

(30) Foreign Application Priority Data
Jul. 1, 2022 (CN) 202210768533.9
Jul. 1, 2022 (CN) 202210768534.3
Jul. 1, 2022 (CN) 202210770077.1
Jul. 1, 2022 (CN) 202210770082.2
Dec. 30, 2022 (CN) 202211732816.4
Mar. 1, 2023 (CN) 202310185951.X

(51) Int. Cl. A61F 2/32; A61F 2/30

(52) U.S. Cl. CPC A61F 2/32; A61F 2002/30616

(57) ABSTRACT

Disclosed are an actuator for hip replacement and a surgical system. The actuator for the hip replacement is configured to prepare a space for installing a prosthesis on a bone and implanting the prosthesis, which includes a first actuator and a second actuator. The first actuator is configured to connect a cutting tool to process an acetabulum and/or a medullary cavity, and the first actuator has a first connector and a second connector. The second actuator is configured to connect to the second connector of the first actuator during an operation of implantation of the prosthesis is performed, and connect the prosthesis and receive impact produced during installing the prosthesis. The actuator for the hip replacement is configured to be installed to a robotic arm via the first connector.

### CROSS-REFERENCE TO RELATED APPLICATIONS

The present application is a continuation of International Application No. PCT/CN2023/103989, filed on Jun. 29, 2023, which claims priority to Chinese Patent Application 202210768533.9, filed on Jul. 1, 2022, Chinese Patent Application 202210768534.3, filed on Jul. 1, 2022, Chinese Patent Application 202210770077.1, filed on Jul. 1, 2022, Chinese Patent Application 202210770082.2, filed on Jul. 1, 2022, Chinese Patent Application 202211732816.4, filed on Dec. 30, 2022, and Chinese Patent Application 202310185951.X, filed on Mar. 1, 2023. The disclosures of the aforementioned applications are hereby incorporated by reference in their entireties.

### TECHNICAL FIELD

The present disclosure relates to the field of medical equipment, and in particular, to an actuator for hip replacement and a surgical system.

### BACKGROUND

Joint replacement surgery mainly comprises knee replacement and hip replacement. In total knee replacement, a distal end of a femur and a tibia that make up a knee joint need to be processed to form a shape and a size suitable for implanting a prosthesis. The processing to the femur and the tibia is mainly made by cutting with a saw, to form a plurality of planes. The precision of the implantation of the prosthesis of the knee joint is substantially determined by the shape of the processed bone, so that processing precision of each plane determines the precision of the implantation of the prosthesis. In total hip replacement, an acetabulum and a proximal end of the femur that make up a hip joint need to be processed to form a shape and a size suitable for implantation of a prosthesis. The forming of the hip joint includes grinding forming of an acetabular fossa, and osteotomy and reaming at a proximal end of the femur. The precision of the hip replacement involves the precision of the implantation of the prosthesis on a side of the acetabulum and the precision of the implantation of the prosthesis on a side of the femur. The precision of the implantation of the prosthesis on the side of the acetabulum depends on the processing precision of the acetabular fossa and control precision of the implanting angle and depth of the acetabular prosthesis during implanting. The precision of the implantation of the prosthesis on the side of the femur depends on the reaming precision on the side of the femur.

When the acetabular fossa is processed, it is necessary to use a grinding tool to grind the acetabular fossa. The grinding tool generally includes a hemispherical file head, a connecting rod with a certain length, a holding sleeve sleeved outside the connecting rod and a pistol-shaped power tool. One end of the connecting rod is connected to the file head, and the other end of the connecting rod is connected to a power output end of the pistol-shaped tool. In use, a surgeon holds a handle of the pistol-shaped power tool in one hand and holds the holding sleeve in the other hand, and then inserts the file head into the acetabulum and applies a force in an axial direction of the connecting rod to grind bone tissue on a surface of the acetabulum. In the grinding process, the surgeon controls an angle between the connecting rod and a pelvis and the depth of the grinding according to experience, to control the processing precision.

After the acetabular fossa is processed, it is necessary to use a cup holding apparatus to implant a prosthesis cup into the acetabulum. The cup holding apparatus includes a straight connecting rod or a connecting rod with an elbow, and a hammer. One end of the connecting rod is connected to the prosthesis cup, and the other end of the connecting rod is used for receiving hitting by the hammer. A middle portion of the connecting rod is used for holding by a surgeon. In use, the surgeon holds the middle portion of the connecting rod to control the angle of the connecting rod with respect to the pelvis. The other end of the connecting rod is hammered by the hammer to press the prosthesis cup into the acetabular fossa. During implanting, with each hammering, the whole cup holding apparatus will move axially with the prosthesis cup entering the acetabular fossa.

MAKO SURGICAL CORP. also provides a surgical robot for the hip replacement, and Chinese Patent Publication CN102612350B discloses its configuration. When the surgical robot is used to grind the acetabulum, it is necessary to first install a grinding tool to a holding structure on an end of the robotic arm, and then a power apparatus is connected to the grinding tool. This holding structure is also used to connect a cup holder to perform an operation for installing a prosthesis. Therefore, after an operation of acetabulum grinding is completed, it is necessary to first remove a power apparatus, and then remove the grinding tool, and finally install the cup holder to the holding structure. The operations in the above-mentioned process are rather complicated.

### SUMMARY

The present disclosure provides an actuator for hip replacement and a surgical system, so that each surgical stage of the hip joint surgery is capable to be performed more conveniently and smoothly.

In the present disclosure, an actuator for hip replacement is provided, which is configured to prepare a space for installing a prosthesis on a bone and implanting the prosthesis, including a first actuator and a second actuator. The first actuator is configured to connect a cutting tool to process an acetabulum and/or a medullary cavity, and the first actuator has a first connector and a second connector. The second actuator is configured to connect to the second connector of the first actuator during an operation of implantation of the prosthesis is performed, and connect the prosthesis and receive impact (also called shock) produced during installing the prosthesis. Where, the actuator for the hip replacement is configured to be installed to a robotic arm via the first connector.

In the present disclosure, a surgical system is provided, including an actuator, a robotic arm, a navigation system and a controlling system. The actuator is the actuator for the hip replacement as described in the first aspect. The robotic arm is connected to the first connector of the actuator. The navigation system is configured to measure a position of the actuator. The controlling system is configured to drive the robotic arm to move the actuator to a target position according to a surgical plan.

The actuator for the hip replacement as provided in the present disclosure includes the first actuator and the second actuator. The first actuator is configured to connect the cutting tool, to process the acetabulum and/or the medullary cavity. The second actuator is connected to the first actuator during the operation of the implantation of the prosthesis is performed, and is configured to connect the prosthesis and receiving the impact produced during installing the prosthesis. During the hip joint surgery, when the acetabulum and the medullary cavity are prepared, the robotic arm is connected to the first actuator. When it is necessary to install the prosthesis, the second actuator is connected to the first actuator. By the above configuration, the operation of actuator replacement is capable to be reduced, thereby facilitating completing the hip replacement surgery.

### BRIEF DESCRIPTION OF THE DRAWINGS

FIG. 1 is a schematic of a surgical system according to an embodiment of the present disclosure.

FIG. 2 is a schematic diagram of an actuator for hip replacement according to an embodiment of the present disclosure.

FIG. 3 is a schematic diagram of usage of a first actuator according to an embodiment of the present disclosure.

FIG. 4 is a structural schematic diagram of a power apparatus according to an embodiment of the present disclosure.

FIG. 5 is a section view of an inner structure of a power apparatus according to an embodiment of the present disclosure.

FIG. 6 is a structural schematic diagram of an output shaft according to an embodiment of the present disclosure.

FIG. 7 is a structural schematic diagram I of a connector and an output shaft according to an embodiment of the present disclosure.

FIG. 8 is a section view of structures of a connector and an output shaft according to an embodiment of the present disclosure.

FIG. 9 is a schematic diagram I of a first type tool assembly according to an embodiment of the present disclosure.

FIG. 10 is a section view of a first type tool assembly according to an embodiment of the present disclosure.

FIG. 11 is a structural schematic diagram of a connecting part according to an embodiment of the present disclosure.

FIG. 12 is a connecting schematic diagram of a rotating joint structure and a spline according to an embodiment of the present disclosure.

FIG. 13 is a section view of structures of a power apparatus and a first type tool assembly according to an embodiment of the present disclosure.

FIG. 14 is a structural schematic diagram of a joint between a power apparatus and a first type tool assembly according to an embodiment of the present disclosure.

FIG. 15 is a schematic diagram of another connecting structure of an output shaft and an adapter shaft according to an embodiment of the present disclosure.

FIG. 16 is a schematic diagram of yet another connecting structure of the output shaft and the adapter shaft according to an embodiment of the present disclosure.

FIG. 17 is a structural schematic diagram of a first actuator, which is connected to a second type tool assembly, according to an embodiment of the present disclosure.

FIG. 18 is a schematic diagram of a whole structure of a second actuator according to an embodiment of the present disclosure.

FIG. 19 is a section view of a whole structure of a second actuator according to an embodiment of the present disclosure.

FIG. 20 is a structural schematic diagram of a joint between a supporting assembly and a sliding rod according to an embodiment of the present disclosure.

FIG. 21 is a schematic diagram of components at an accommodating channel according to an embodiment of the present disclosure.

FIG. 22 is a schematic diagram of a second actuator installed by a first actuator according to an embodiment of the present disclosure.

FIG. 23 is a structural schematic diagram I of a supporting assembly and a second connector according to an embodiment of the present disclosure.

FIG. 24 is a structural schematic diagram II of the supporting assembly and the second connector according to an embodiment of the present disclosure.

FIG. 25 is a structural schematic diagram III of the supporting assembly and the second connector according to an embodiment of the present disclosure.

FIG. 26 is a structural schematic diagram of a sliding rod, which is installed with an adjusting member, according to an embodiment of the present disclosure.

FIG. 27 is a schematic diagram I of an adjusting member according to an embodiment of the present disclosure.

FIG. 28 is a schematic diagram II of the adjusting member according to an embodiment of the present disclosure.

FIG. 29 is a schematic diagram III of the adjusting member according to an embodiment of the present disclosure.

FIG. 30 is a structural schematic diagram of a nut according to an embodiment of the present disclosure.

FIG. 31 is a structural schematic diagram I of a prosthesis installing actuator as provided in an embodiment of the present application.

FIG. 32 is a section view of a prosthesis installing actuator as provided in an embodiment of the present application.

FIG. 33 is a schematic diagram of a first damping mechanism.

FIG. 34 is a three-dimensional structural schematic diagram II of the prosthesis installing actuator as provided in an embodiment of the present application.

### DETAILED DESCRIPTION OF THE EMBODIMENTS

Hereinafter, features and exemplary embodiments of various aspects of the present disclosure will be described in detail. In order to make purposes, technical solutions and advantages of the present disclosure clearer and more understandable, the present disclosure will be further described in detail in combination with the drawings and specific embodiments as below. It should be understood that, the specific embodiments as described herein are intended merely to explain the present disclosure, rather than limiting the present disclosure. For a person skilled in the art, the present disclosure may be implemented without some of the specific details. The description for the embodiments as below is intended merely to provide better understanding to the present disclosure by illustrating examples of the present disclosure.

It should be noted that, in the present specification, the relation terms, such as “first” and “second” or the like, are used only to distinguish one entity or operation from another entity or operation, rather than requiring or implying that there is any actual relationship or order between these entities or operations. Moreover, the terms “comprise”, “include” or any other variations thereof is intended to cover non-exclusive inclusion, such that any process, method, item or device including a series of elements not only includes said elements, but also further includes other element(s) not specifically listed, or further includes the element(s) inherent to the process, method, item or device. In the cases without any more limitation(s), the element(s) as defined by a sentence of “include . . . ” will not exclude the case(s) in which the process, method, item or device including said elements further includes additional same element(s).

It should be noted that, the embodiments and the features therein in the present disclosure may be combined with one another, unless it causes contradiction. Hereinafter, the embodiments will be described in detail in combination with the drawings.

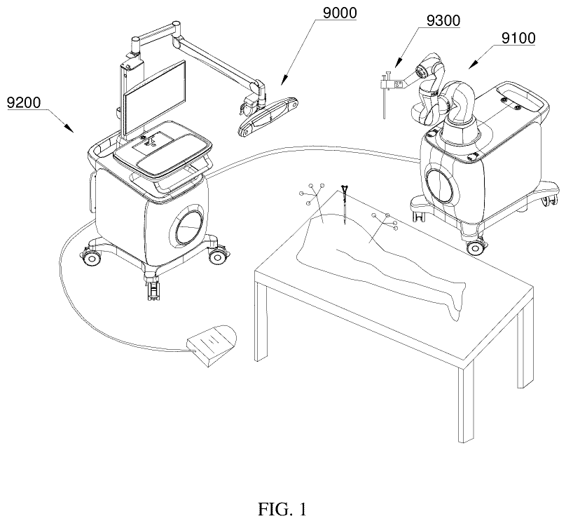

As shown in FIG. 1, a robotic system as provided in the present disclosure includes a robotic arm 9100, a navigation system 9000, an actuator for hip replacement 9300 and a controlling system 9200. The robotic arm 9100 corresponds to an arm of a surgeon, and it may hold a surgical tool and position the surgical tool in a relatively high precision. The navigation system 9000 corresponds to an eye of the surgeon, and it may measure a position of the surgical tool and a tissue of a patient in real time. The controlling system 9200 corresponds to a brain of the surgeon, and it stores a surgical plan therein. The controlling system 9200 calculates a path for the robotic arm and/or its position to be reached according to information obtained by the navigation system 9000 during surgery, and may actively control movement of the robotic arm 9100, or configure a virtual boundary for the robotic arm 9100 through a force feedback mode, and then manually push the robotic arm 9100 to move along a path or face as defined by the virtual boundary or within a defined volume.

The actuator for the hip replacement 9300 is configured to prepare a space for installing a prosthesis on a bone and implanting the prosthesis. The actuator for the hip replacement includes a first actuator and a second actuator. The first actuator is configured to connect a cutting tool to process an acetabulum and/or a medullary cavity. The first actuator has a first connector and a second connector. The second actuator is configured to connect to the second connector of the first actuator during an operation of implantation of the prosthesis is performed, and for connecting the prosthesis and receiving impact (also called shock) produced during installing the prosthesis. The actuator for the hip replacement is configured to be installed to a robotic arm 9100 via the first connector. In a hip joint surgery, when it is to process the acetabulum and the medullary cavity, the first actuator is connected onto the robotic arm 9100; and when it is to install the prosthesis, the second actuator is connected to the first actuator. By the above configuration, the operation of replacing the actuator is capable to be reduced.

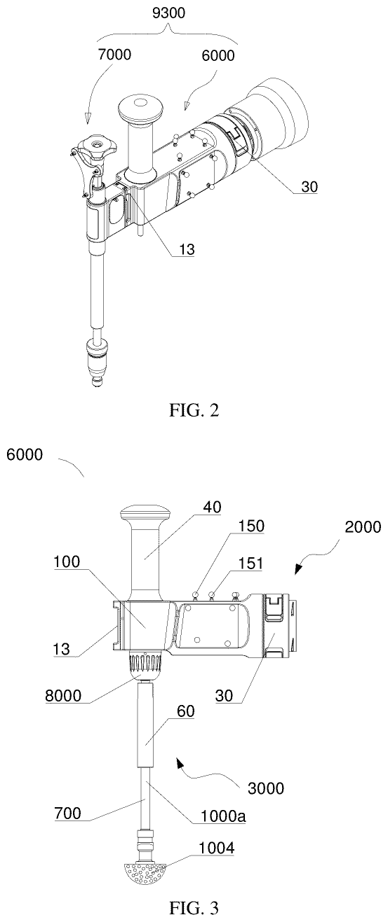

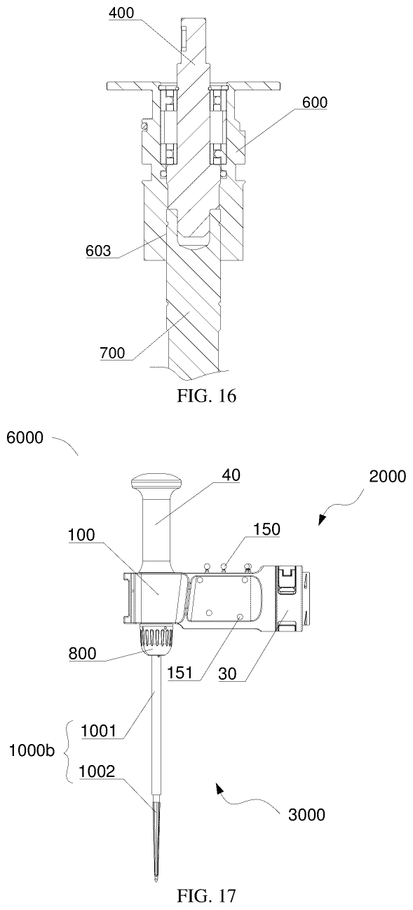

Specifically, in the hip replacement, after a hip joint at an affected area is exposed, it is generally to firstly prepare an acetabular fossa. During this process, it is necessary to use a rotating acetabulum file to grind the acetabular fossa at the affected area, to prepare a shape suitable for installing the prosthesis. FIG. 1 shows a schematic diagram of usage of the first actuator for preparing the acetabular fossa. The first actuator 6000 is connected to the robotic arm 9100 through the first connector 30, the first actuator 6000 is detachably connected to an acetabulum file tool assembly (for example a surgical tool 1000a), and an end of the acetabulum file tool assembly is configured to connect a file head (for example a cutting tool 1004). In this case, the acetabulum file tool assembly may be driven by the first actuator 6000 to grind the acetabulum. After the acetabulum is prepared, it is necessary to place the prosthesis into the acetabulum. FIG. 2 shows a state in which the second actuator 7000 is connected to the second connector 13 of the first actuator 6000 (at this time, the acetabulum file tool assembly on the first actuator 6000, used for grinding the acetabulum, is removed). The second actuator 7000 is connected to the robotic arm 9100 indirectly through the first actuator 6000, and the prosthesis may be installed, under holding of the robotic arm 9100. Further, as shown in FIG. 17, if it is to perform reaming on a proximal end of a femur, it is necessary to remove the second actuator 7000 from the second connector 13, and install a medullary cavity reamer assembly (for example a surgical tool 1000b) for reaming onto the first actuator 6000.

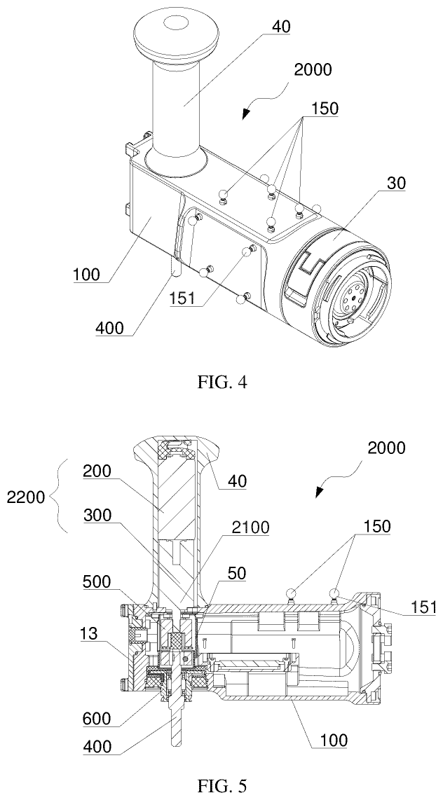

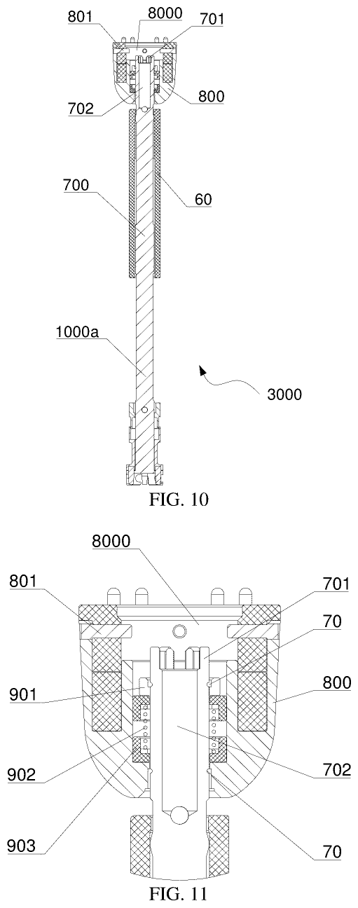

Hereinafter, the first actuator 6000 will be described. A tool assembly 3000 therein is a connecting rod for connecting the acetabulum file head, as shown in FIGS. 1 to 17.

The first actuator 6000 is a joint forming actuator, which is configured to prepare the formed acetabular fossa or medullary cavity on a hip joint. The first actuator 6000 includes a power apparatus 2000 and a tool assembly 3000. The power apparatus 2000 includes a housing 100 and a built-in power assembly 2100. The first actuator 6000 is connected to an end of the robotic arm 9100 of the robot. The power assembly 2100 includes a power source 2200 and an output shaft 400, and the output shaft 400 is connected to the power source 2200. The tool assembly 3000 includes a connecting part 8000 and a surgical tool 1000a, and the surgical tool 1000a is rotatably provided on the connecting part 8000. The tool assembly 3000 is detachably provided on the power apparatus 2000 through the connecting part 8000. When the tool assembly 3000 is connected to the power apparatus 2000 via the connecting part 8000, the surgical tool 1000a is joined the output shaft 400 to receive rotation movement output by the output shaft 400. The power assembly 2100 is provided in the housing 100 and outputs power through the output shaft 400. The output shaft 400 is engaged with an end of the tool assembly 3000 to drive a connecting rod of the grinding file, without using a long guiding cylinder to guide the connecting rod, so that a structure of the actuator is more compact. In this way, interference and influence on a surgery space and influence on safety due to an externally connected power source is reduced, and operations of assembling the externally connected power source during surgery is reduced, and thus the surgery process is smoother.

Specifically, as shown in FIGS. 2, 4 and 5, the first actuator 6000 includes a power apparatus 2000 and a tool assembly 3000. The power apparatus 2000 includes a housing 100 and a power assembly 2100. The housing 100 is an internal hollow part with a generally quadrangular shape. The housing 100 is provided, at its two ends, with a first connector 30 and a second connector 13, respectively. The first connector 30 is used as a connecting end to connect the first actuator 6000 to the robotic arm 9100. The second connector 13 is used as a connector for a prosthesis installing actuator, and is used for detachably connecting the prosthesis installing actuator (for example a second actuator 7000). The housing 100 is further provided thereon with a handle 40. The handle 40 is hollow inside, and is detachably connected to the housing 100. A structure of the power apparatus 2000 used for connecting the tool assembly 3000 is a quick joining joint, which is provided on another side of the housing 100 opposite to the position of the handle 40. When the tool assembly 3000 is installed to the quick joining joint, an axis of the handle 40 and an axis of the surgical tool 1000a are basically in a straight line, and they are arranged on two sides of the power apparatus 2000. Each surface of the housing 100 is used for connecting a tracing assembly 150 to indicate a position of the actuator.

As shown in FIG. 5, the power assembly 2100 includes a motor 200, a speed reducer 300, an output shaft 400 and a coupling 500. The motor 200 and the speed reducer 300 constitute the power source 2200. The power source 2200 is integrated in the handle 40, and is fixedly connected to the housing 100. The speed reducer 300 includes a shaft connected to the output shaft 400 via the coupling 500. The power source 2200 and the output shaft 400 are provided coaxially, with the axes thereof perpendicular to the housing 100.

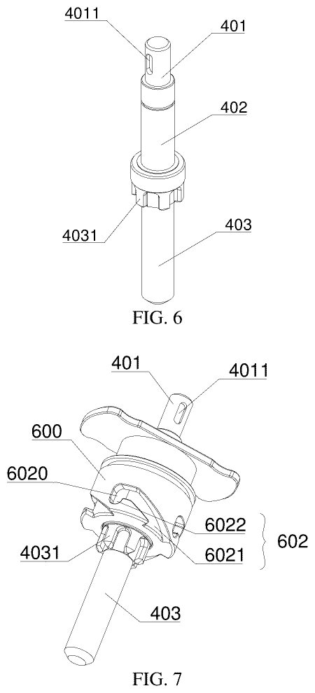

As shown in FIG. 6, the output shaft 400 includes an input section 401, a middle section 402 and an output section 403 which are provided in sequence. The input section 401 is provided thereon with a keyway 4011 for receiving rotation movement from the power source 2200. The middle section 402 is installed in a bearing in the power apparatus 2000. The output section 403 is provided with a coupling spline 4031 which includes a plurality of protrusions distributed at intervals in a circumferential direction, and is configured to output a torque. A length of the coupling spline 4031 is less than a length of the output section 403. That is, the last section of the output section 403 is a smooth shaft.

Referring to FIGS. 5 to 8 herewith, the housing 100 is further provided thereon with a joint 600 fixed to the housing 100.

The joint 600 is used for connecting the tool assembly 3000 and installing the output shaft 400. The joint 600 has a columnar shape, and is provided therein with a hole 601 and is provided with four spiral grooves 602 on its periphery. The spiral groove 602 is used for guiding a pin shaft member and includes a limiting part 6020 for limiting an axial direction and a circumferential direction of the pin shaft member. An end of the joint 600 is provided with two wing plates along a radial direction. The hole 601 is used for installing a bearing therein and accommodating the middle section 402 of the output shaft 400. The spiral groove 602 includes a rotating section 6021 and a positioning section 6022. The rotating section 6021 spirally extends in a first axial direction, the positioning section 6022 extends from an extending end of the rotating section 6021 towards a second axial direction, and the second axial direction is opposite in direction to the first axial direction. A side wall of the positioning section 6022 forms the limit portion 6020. The positioning section 6022 is used to form limit in the second axial direction and limit in a circumferential direction to an object contained in the groove. The wing plate is used for fixing the joint 600 with the housing 100. When the output shaft 400 is installed to the joint 600, the coupling spline 4031 extends out of the hole 601 and is located outside the housing 100.

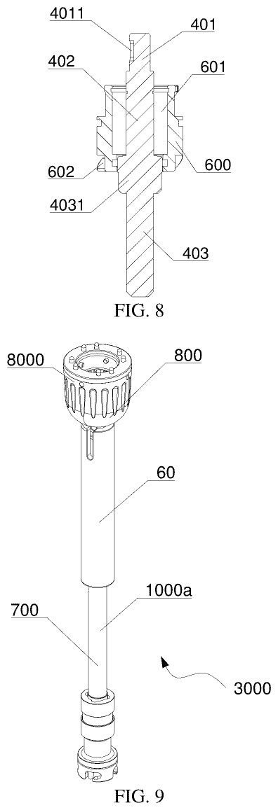

As shown in FIGS. 9 to 11, the tool assembly 3000 includes a connecting part 8000 and a surgical tool 1000a. The surgical tool 1000a is rotatably provided, by its one end, on the connecting part 8000. The surgical tool 1000a is an acetabulum grinding file rod assembly, and is connected, by its other end, with the acetabulum grinding file. The acetabulum grinding file rod assembly includes a connecting rod main shaft 700, an acetabulum file connecting component and a holding sleeve 60. An end of the connecting rod main shaft 700 is rotatably connected to the connecting part 8000, and the other end of the connecting rod main shaft 700 is provided with the acetabulum file connecting component. The holding sleeve 60 is sleeved outside the connecting rod main shaft 700. An end of the connecting rod main shaft 700 connected to the connecting part 8000 is provided with a spline joint 701 and an joint hole 702. The spline joint 701 is capable to be engaged and matched with the coupling spline 4031, to achieve transmission of the rotation movement. But they are not closely fitted, and may be separated in an axial direction. A diameter of the joint hole 702 is equal to a diameter of the smooth shaft portion of the output section 403.

The connecting part 8000 includes a connecting rod lock head 800 and a connecting rod connection module. The connecting rod lock head 800 has an internal hollow cup shape, and is provided with a circular hole on its bottom. The connecting rod lock head 800 is provided, in positions on its inner circumferential face close to an opening, with four positioning pins 801 distributed circumferentially. The connecting rod connection module is provided in the connecting rod lock head 800, and is used for rotatably connecting the acetabulum grinding file rod assembly to the connecting rod lock head 800.

The connecting rod connection module includes a clamping support 901, a positioning module and a pair of pad sleeves 903, which are coaxially held in the connecting rod lock head 800. The clamping support 901 is in a ring shape and is provided on the outermost side (a side where the opening of the connecting rod lock head 800 is located). The positioning module includes an elastic member 902, and is configured to form a predetermined acting force between the connecting part 8000 and the power apparatus 2000. In the present embodiment, the elastic member 902 is a thrust spring. Both the two pad sleeves 903 are in a ring shape, and are provided axially between the clamping support 901 and a bottom of the connecting rod lock head 800. An outer circumferential face of the pad sleeve 903 is fitted with an inner circumferential face of the connecting rod lock head 800, and a diameter of an inner hole of the pad sleeve 903 is equal to a diameter of the connecting rod main shaft 700. The thrust spring is provided between the two pad sleeves 903.

The connecting rod main shaft 700 is sleeved in the clamping support 901, the thrust spring and the pad sleeves 903. A peripheral face of the connecting rod main shaft 700 is further provided with two ring grooves 70 having a predetermined distance, and the ring grooves 70 are used for installing baffle rings. With the assembling relation, the clamping support 901, the thrust spring, the pad sleeves 903 and the connecting rod lock head 800 are all located between the two baffle rings. Therefore, the connecting rod lock head 800 and the connecting rod main shaft 700 form an integrated structure. The thrust spring may be compressed, thus allowing a certain amount of movement of the connecting rod lock head 800 in an axial direction of the connecting rod main shaft 700.

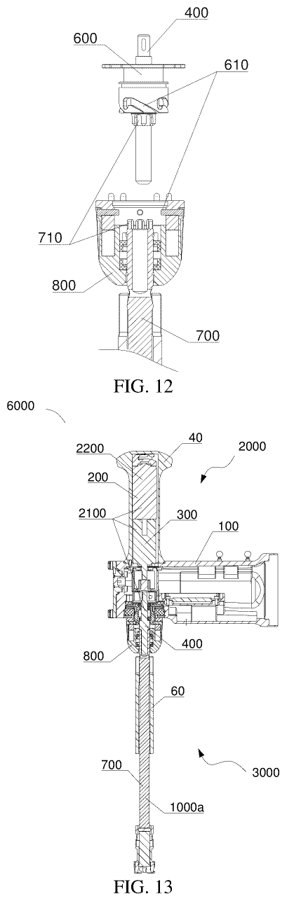

As shown in FIG. 12, the connecting part and the power apparatus 2000 will be connected by a rotating joint structure 610 to form an axial and circumferential limit to the connecting part. Herein, the rotating joint structure 610 includes a positioning pin 801 and a spiral groove 602. That is, the tool assembly 3000 is connected to the housing 100 through the positioning pin 801 being rotating joint and fitting with the spiral groove 602.

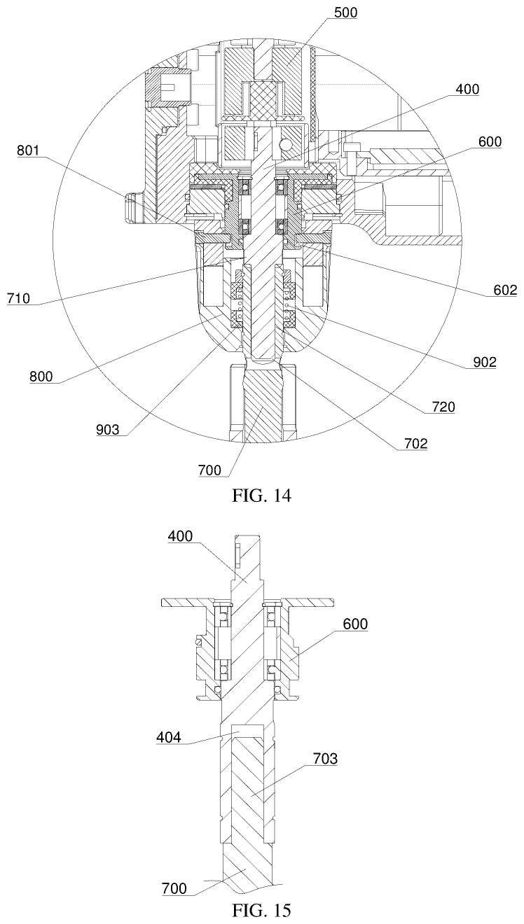

FIGS. 13 and 14 show structural schematic diagrams of an acetabulum grinding file rod assembly installed to the power apparatus 2000. With the assembling relation, the positioning pin 801 is inserted and connected in the positioning section 6022 of the spiral groove 602. Two side walls of the positioning section 6022 extending in an axial direction form the circumferential limit to the positioning pin 801, and an end wall of the positioning section 6022c forms the axial limit to the positioning pin 801. Therefore, if there is no external force applied, the connecting rod lock head 800 will not drop axially or rotate circumferentially. The connecting part 8000 forms radial positioning with the connecting rod main shaft 700 and the housing 100, which is equivalent to that the radial positioning is formed between the connecting rod main shaft 700 and the output shaft 400 (which is positioned on the housing 100). Specifically, referring to FIG. 12 and FIG. 14, the smooth shaft portion of the output shaft 400 and the joint hole 702 of the connecting rod main shaft 700 form a radially positioning structure 710. The radially positioning structure 710 is a shaft-hole fit structure, with equal diameters. That is, a direct radial positioning is formed between the output shaft 400 and the joint hole 702. With limitation to a length and a fitting precision of a fitting section for forming the radial positioning between the connecting part 8000 and the connecting rod main shaft 700, a certain amount of radial movement of the connecting rod main shaft 700 may be possible. The radial positioning between the smooth shaft portion of the output shaft 400 and the joint hole 702 of the connecting rod main shaft 700 is capable to improve precision of the radial positioning. The spline joint 701 of the connecting rod main shaft 700 is aligned and engaged with the coupling spline 4031 of the output shaft 400 to receive the rotation movement. The axial acting force applied by the thrust spring to the connecting rod lock head 800 causes the positioning pin 801 to be pressed axially against the end wall of the positioning section 6022. As the thrust spring is compressed, there is an internal stress in the connection between the connecting part 8000 and the power apparatus 2000. Such internal stress enables stable axial positioning to be formed between the tool assembly 3000 and the power source, without increasing difficulty of design or difficulty of installation to ensure the precision of the axial positioning, and thus provides more stable connection and avoids loosening due to vibration or the like. Moreover, the connecting rod main shaft 700 is pushed axially by the thrust spring against the output shaft 400 to form axial positioning.

Compared with thread screwing connection, the cooperation of the positioning pin 801 and the spiral groove 602 is labor-saving, facilitating fast assembling/disassembling during surgery. The direct physical limit of the positioning section 6022 to the positioning pin 801 is more reliable than frictional locking. In some optional embodiments, the positioning pin 801 may be provided on the outer circumferential face of the connecting rod lock head 800, and the spiral groove 602 may be provided on the inner circumferential face of the joint 600. In other optional embodiments, the positioning pin 801 may be provided on the inner/outer circumferential face of the joint 600, and the spiral groove 602 may be provided on the outer/inner circumferential face of the connecting rod lock head 800. By such configuration, it also ensures that the positioning pin 801 and the spiral groove 602 are capable to be rotated and joined when they cooperate with each other, and further the axial and circumferential positioning of the joint 600 and the connecting rod lock head 800 is enabled.

The joint between the output shaft 400 and the connecting rod main shaft 700 is achieved by a spline connection 710 in which during joining, it is only necessary to make the connecting rod main shaft 700 align the output shaft 400 axially, convenient in operation. In some optional embodiments, it is also possible to form connection allowing torque transmission between the output shaft 400 and the connecting rod main shaft 700 by mutual embedding of end faces.

As shown in FIG. 15, in some optional embodiments, it is possible to use another alternative radially positioning structure for the radial positioning between the smooth shaft portion of the output shaft 400 and the joint hole 702 of the connecting rod main shaft 700. For example, a positioning shaft 703 is provided at an end of the connecting rod main shaft 700, and a positioning hole 404 is provided on the output shaft 400, thus forming the radial positioning by the shaft-hole fit thereof. Or, as shown in FIG. 16, a shaft-hole fit structure is provided between the connection joint 600 and the connecting rod main shaft 700. For example, a hole 603, having a diameter larger than a diameter of the spline portion of the output shaft 400, is provided at an end of the joint 600, and accordingly an end of the connecting rod main shaft 700 is configured to have the same diameter, thus forming the shaft-hole fit therebetween.

In some optional embodiments, it is also possible to provide a spring in other positions, functioning as the elastic member 902 in the positioning module, to form the internal stress between the tool assembly 3000 and the power apparatus 2000. For example, a pressure spring is fixed on the power apparatus 2000. When the tool assembly 3000 is installed to the power apparatus 2000, the connecting rod lock head 800 compresses the pressure spring, and the reaction force of the pressure spring tightly presses the positioning pin 801 of the connecting rod lock head 800 in the spiral groove 602, such that a pre-pressure is maintained between the connecting rod lock head 800 and the power apparatus 2000, forming relatively stable connection. In a state of final use, the connecting rod main shaft 700 will be applied with a reaction force by tissue of a patient so as to be axially pressed against the output shaft. The pressure spring may include general a coil spring, a disc spring, a wave spring and the like. Certainly, the elastic member 902 is not limited to the form of springs, and it may be an elastic piece.

Hereinafter, the use process of the hip joint former (that is, the first actuator 6000) will be specifically explained.

In use, the first actuator 6000 is connected, via the first connector 30, with the robotic arm 9100, where at this time, the first actuator 6000 is not installed with the tool assembly 3000. Firstly, the robotic arm 9100 comes into the ready position according to the predetermined surgical plan. A surgeon installs the acetabulum grinding file rod assembly, provided with the acetabulum file (for example the cutting tool 1004), to the first actuator 6000 via the joint 600. Specifically, the surgeon holds the connecting rod lock head 800 in hand, axially sleeves the joint hole of the connecting rod main shaft 700 to the output section 403 of the output shaft 400, and makes the coupling spline 4031 be aligned and joined with the spline joint 701. After the output shaft 400 is circumferentially joined with the connecting rod main shaft 700, the connecting rod main shaft 700 is connected against the output shaft 400. The surgeon pulls and rotates the connecting rod lock head 800 in a direction close to the actuator, such that the positioning pin 801 of the connecting rod lock head 800 moves in the spiral groove 602 along the rotating section 6021 and finally into the positioning section 6022.

Thus, the joint of the coupling spline 4031 and the spline joint 701 results in the circumferential joint of the output shaft 400 and the connecting rod main shaft 700, and the cooperation of the output section 403 and the joint hole 702 improves coaxiality of the connection, thus, together with the connecting rod lock head 800, increasing a length of the radial positioning to the connecting rod main shaft 700, and thus improving the coaxiality of the output shaft 400 and the connecting rod main shaft 700 during transmission of rotation. When the positioning pin 801 is in the positioning section 6022, the positioning pin 801 cannot circumferentially rotate with respect to the joint 600 due to limitation by the two side walls of the positioning section 6022 extending axially. The thrust spring is configured such that there is a trend for the connecting rod lock head 800 to move towards the connecting rod main shaft 700 with respect to the joint 600. Such moving trend prevents the positioning pin 801 from moving axially out of the positioning section 6022 to reach the rotating section 6021. The thrust spring makes the connecting rod main shaft 700 press axially against the output shaft 400. That is, the thrust spring pushes the connecting rod main shaft 700 to keep joining the output shaft 400 axially. In the above operation process, the radially positioning portion of the connecting rod main shaft 700 is its top end. The acetabulum grinding file rod assembly moving axially has a relatively small stroke, and thus the needed operation space is accordingly relatively small.

By now, the acetabulum grinding file rod assembly is connected to the housing 100. With the guidance of the predetermined surgical plan, the first actuator 6000, under control of the robotic arm 9100 and the surgeon, is moved to the predetermined target position. The motor 200 is started and the rotation thereof is transmitted in sequence to the speed reducer 300, the coupling 500 and the output shaft 400. As the output shaft 400 and the connecting rod main shaft 700 are connected via the coupling spline 4031 and the spline connection joint 701, the connecting rod main shaft 700 is driven by the output shaft 400 to rotate. In the process of such rotation, as the connecting rod lock head 800 and the joint 600 are fixedly connected, the connecting rod lock head 800 will not rotate. The rotating connecting rod main shaft 700 drives the acetabulum file (the cutting tool 1004) to rotate, to perform grinding and forming of the acetabular fossa.

After grinding forming of the acetabular fossa is completed according to the predetermined surgical plan, the robotic arm 9100 comes into a posture by which the acetabulum grinding file rod assembly may be disassembled. The surgeon overcomes the elastic force of the thrust spring and pulls the connecting rod lock head 800, such that the positioning pin 801 moves out of the limitation by the positioning section 6022, then rotates the connecting rod lock head 800, and the positioning pin 801 passes the rotating section 6021 and then is detached from the spiral groove 602, and thus the connecting rod lock head 800 is detached from the joint 600. The acetabulum grinding file rod assembly is moved away from the joint 600 in the axial direction of the connecting rod main shaft 700, thus completing disassembling.

In sum of above, the motor 200, the speed reducer 300, the coupling and the output shaft 400 are integrated in the housing 100. A power line of the motor 200 may be introduced by a connector between the housing 100 and the robotic arm 9100. The first actuator 6000 has a compact structure, without providing an external connected power source, and thus avoiding the intervention and influence of the externally connected power source and its power line on the surgery space as well as the safety risk due to exposure of the power line. As it is not necessary to assemble an externally connected power source during surgery, the operation steps of the surgery are reduced. The tool assembly 3000 includes a connecting part 8000 and the acetabulum grinding file rod assembly, and as a pre-assembled modular part, it may be used to achieve removable connection between the surgical tool 1000a and the output shaft 400 conveniently.

As shown in FIG. 17, in an optional embodiment, the surgical tool 1000b is a medullary cavity reamer, and the tool assembly 3000 includes the connecting part 8000 and the medullary cavity reamer. The medullary cavity reamer includes a reamer rod 1001 and a reamer, the reamer is used for reaming and is connected to the reamer rod 1001. The reamer rod 1001 is provided at its end with the spline joint 701, the spline joint 701 is used for connecting with the coupling spline 4031. The reamer is provided thereon with a reamer blade 1002, for performing reaming on a medullary cavity of a femur under rotation movement. The connecting part 8000 is same in structure as the above-described connecting part 8000 connected to the acetabulum grinding file rod assembly. The connecting rod connection module connects the reamer rod 1001 with the connecting rod lock head 800. Moreover, the tool assembly 3000 connected to the medullary cavity reamer is also connected to the joint 600 and the output shaft 400 in the same way. After the connecting rod lock head 800 is connected to the joint 600, the medullary cavity reamer uses the spline key connection joint 701 to join the coupling spline key 4031, and thus to the output shaft 400. The output shaft 400 is driven by the motor 200 to drive the medullary cavity reamer to rotate and perform the task of reaming at the proximal end of the femur.

In an optional embodiment, the first actuator 6000 is provided with three groups of tracing assemblies 150. The three groups of tracing assemblies 150 are provided on three surfaces of the housing 100, respectively. Each group includes four tracing elements 151 located on the same plane. As shown in FIGS. 2 to 4, the housing 100 is provided thereon with three planes, and the three groups of the tracing elements 151 are provided on the three planes, respectively. The tracing element 151 may be a passive reflective ball or reflective sheet, or may be an active electromagnetic generator or sensor.

It is understandable that, during a hip joint forming surgery, the tracing element 150 sends position information of the first actuator 6000 to a positioner. The positioner, generally in a constant position in the surgery space, is an apparatus in the navigation system 9000 for receiving position information. By providing three groups of the tracing elements 151, the position information of the first actuator 6000, even in various postures, is capable to be identified. Corresponding to the tracing elements 151, the positioner may be an optical navigator for identifying reflected light, or may be a receiver for identifying an electromagnetic signal.

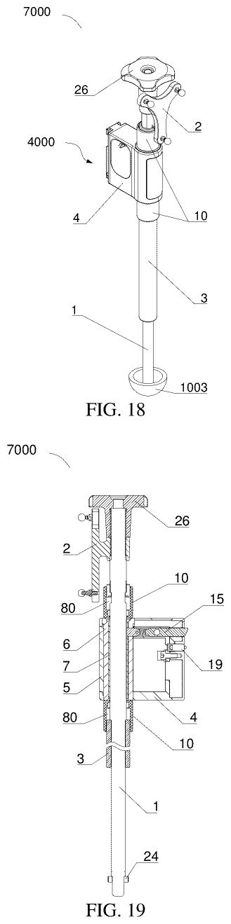

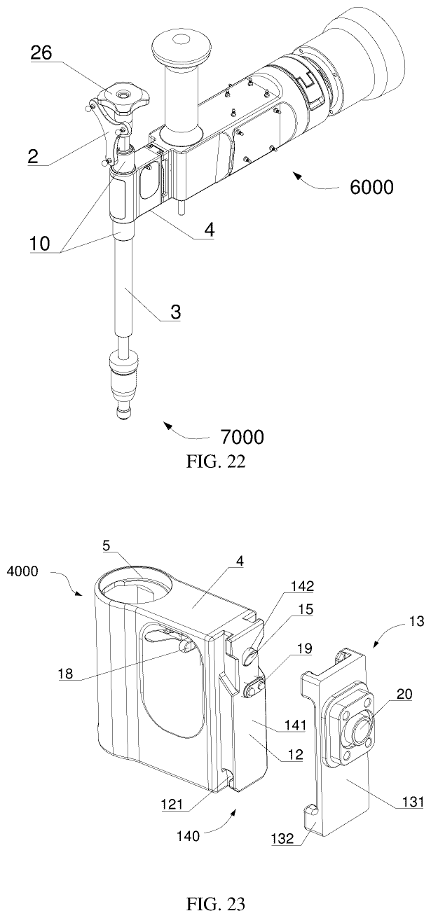

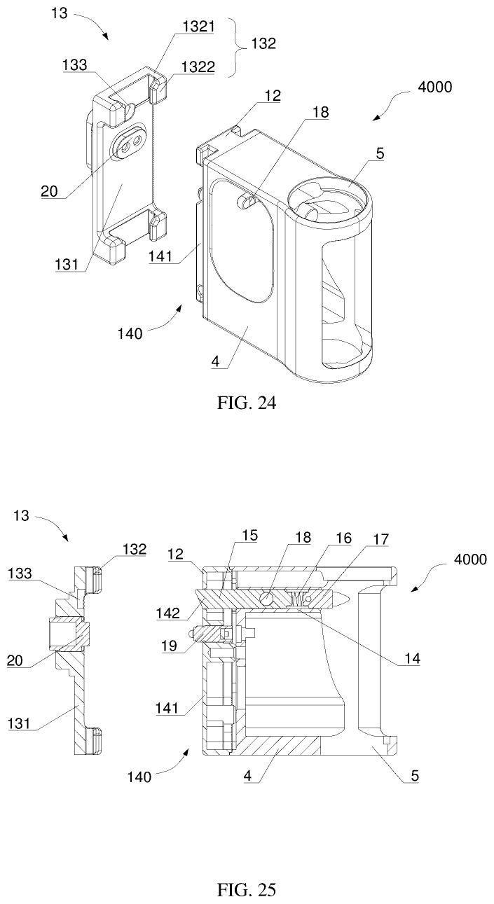

Hereinafter, the second actuator 7000 will be specifically described, as shown in FIGS. 18 to 25.

The second actuator 7000 is a prosthesis installing actuator, for installing a prosthesis 1003 during a hip replacement surgery. The prosthesis installing actuator includes a sliding rod 1, a supporting assembly 4000 and a tracer 2. A first end of the sliding rod 1 is used for connecting the prosthesis 1003, and a second end of the sliding rod 1 is configured to receive the impact produced during installation of the prosthesis. The supporting assembly 4000 includes an accommodating channel 5 accommodating a portion of a rod section of the sliding rod 1, and the sliding rod 1 is axially movable with respect to the supporting assembly 4000. The supporting assembly 4000 is used for connecting the second actuator 7000 to the robotic arm 9100 of the robot system. The tracer 2 is provided on the sliding rod 1 to indicate the orientation of the sliding rod 1. In the second actuator 7000, the sliding rod 1 is axially movable with respect to the supporting assembly 4000. In use, an axial gap between the sliding rod 1 and the supporting assembly 4000 may be made larger than a stroke of the sliding rod 1 when the sliding rod 1 is hit, to prevent the sliding rod 1 from colliding with the supporting assembly 4000 and damaging the robotic arm 9100 connected to the actuator. The sliding rod 1 and the supporting assembly 4000 are configured as an integrated structure. When the actuator is to be used, it is not necessary to assemble or disassemble the sliding rod 1 and the supporting assembly 4000, and it is only necessary to use the supporting assembly to connect the whole actuator to the robotic arm 9100 or remove it from the robotic arm 9100.

Specifically, in embodiments as shown in FIGS. 3 and 18 to 25, the second actuator 7000 includes a sliding rod 1, a supporting assembly 4000, a tracer 2, an axial buffering mechanism 80 and an axial limit structure 90. The second actuator 7000 is connected to the first actuator 6000 through the supporting assembly 4000, and when the first and second actuators are connected, the sliding rod 1 of the second actuator 7000 is parallel to a structure of the first actuator 6000 for connecting the cutting tool 1004. The structure of the first actuator 6000 for connecting the cutting tool 1004 includes the output shaft 400 and the joint 600, with their axes parallel to the sliding rod 1. Both the forming of the acetabular fossa/femur medullary cavity and the implantation of the prosthesis relate to a precision of an angle of the axis of the tool, and the precision of the angle of the axis is inter-related. It is more advantageous to configure the structure for connecting the cutting tool 1004 to be parallel to the structure for connecting the prosthesis 1003.

As shown in FIGS. 18 to 21, the sliding rod 1 is a metal rod having a smooth surface. One end of the sliding rod 1 is configured to receive hammering by a surgeon, and the other end of the sliding rod 1 is used for connecting the prosthesis 1003. The sliding rod 1 is provided, at its middle portion, with a holding part 3. The holding part 3 is in a sleeve shape, and is sleeved on the sliding rod 1 and fixed with the sliding rod 1, to make the surgeon is capable to hold the sliding rod 1 through the holding part 3. The holding part 3 is an insulating plastic sleeve. The sliding rod 1, as a metal member, ensures a relatively high strength during transmitting of the impact. However, an over-heavy surgical instrument is not desirable, and thus the sliding rod 1 generally has a relatively small diameter, such that it is not convenient for the surgeon to hold it. The holding part 3 with the plastic material increases the diameter in the holding position of the sliding rod 1 to provide an advantageous holding condition for the surgeon, without adding a relatively large weight to the surgical tool. Certainly, in some embodiment, the holding part 3 may be an insulating rubber sleeve or a non-insulating metal sleeve. In further other embodiments, it is also possible to provide the holding part 3 without the sleeve shape, and the holding part 3 may be configured as a portion of the sliding rod 1 itself, and such portion may be larger in diameter than the diameter of a main body of the sliding rod 1 to facilitate holding.

The tracer 2 includes a tracing portion and a connecting portion. The tracing portion is provided with a plurality of positioning labels for providing position information. The positioning label may be a reflective ball or a reflective sheet which can reflect infrared light, or may be an infrared light source or an electromagnetic generator which can actively send a signal to achieve positioning. The connecting portion is used for fixing the tracer 2 to the sliding rod 1.

The supporting assembly 4000 includes a supporting member 4, an accommodating channel 5, an insulating sleeve 6 and a sliding sleeve 7. The supporting member 4 is generally in a hexahedron shape, with an end (such as a right end shown in FIG. 19) for connecting the robotic arm 9100. The accommodating channel 5 is a hole penetrating through the supporting member 4. The insulating sleeve 6 and the sliding sleeve 7 are each in a cylinder shape. The insulating sleeve 6 is sleeved in the accommodating channel 5 and is axially fixed to the accommodating channel 5. The insulating sleeve 6 is configured to avoid forming a conductive path between a patient and the device(s) of the robotic arm 9100 due to contact of the supporting assembly 4000 and the sliding rod 1. The sliding sleeve 7 is sleeved in the insulating sleeve 6, and is axially fixed to the insulating sleeve 6. The sliding sleeve 7 is made of metal. The sliding rod 1 and the sliding sleeve 7 form a shaft-hole fit, and there is a gap between the sliding rod 1 and the sliding sleeve 7 that allows the sliding rod 1 to slide freely with respect to the sliding sleeve 7. Thus, the sliding sleeve 7 provided between the insulating sleeve 6 and the sliding rod 1 may not only reduce abrasion of the insulating sleeve 6, but also increase smoothness of sliding of the sliding rod 1.

The axial limit structure 90 includes a check ring 9, and a first end on the holding part 3 away from the prosthesis 1003. Both the check ring 9 and the first end of the holding part 3 are fixed to the sliding rod 1, and two steps with diameters larger than a diameter of sliding rod 1 are formed on the sliding rod 1. When the sliding rod 1 moves along the sliding sleeve 7, the intervention occurs between the two steps and the supporting assembly to form axial limit to the sliding rod 1. In the present embodiment, an insulating member 10 is further provided between the check ring 9 and the supporting assembly 4000 and between the holding part 3 and the supporting assembly 4000. Therefore, actually the check ring 9 and the holding part 3 directly form axial intervention with the insulating member 10. The insulating member 10 is a sleeve with both ends opened. The insulating member 10 includes an inner space having a diameter larger than the diameter of the sliding rod 1. A diameter of an opening at an end of the insulating member 10 is larger than the diameter of the sliding rod 1. A diameter of an opening at the other end of the insulating member 10 is equal to the diameter of the sliding rod 1, and this end is provided with a check rim 101 to form the opening having the same diameter as that of the sliding rod 1. When the sliding rod 1 is assembled to the supporting assembly 4000, the check ring 9 and the first end of the holding part 3 are located on both sides of the supporting assembly 4000, respectively. Both the two insulating members 10 are sleeved on the sliding rod 1, and also are located on both sides of the supporting assembly 4000, respectively. A side of the insulating member 10 having the check rim 101 is connected to the supporting member 4. Thus, the check ring 9 and the first end of the holding part 3 form two limit points on the sliding rod 1. When the sliding rod 1 slides with respect to the supporting assembly 4000, the check ring 9 and the first end of the holding part 3 limit a maximum sliding stroke of the sliding rod 1 with respect to the supporting assembly 4000.

In an optional embodiment, the first end of the holding part 3 in the axial limit structure 90 may be substituted by an independently arranged check ring 9. In another optional embodiment, the check ring 9 or the first end of the holding part 3 may be a step or shoulder provided on the sliding rod 1.

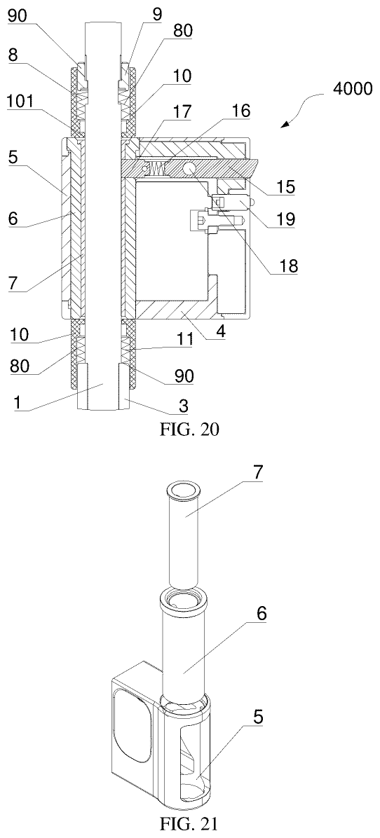

Specifically, referring to FIGS. 19 and 20, an axial buffering mechanism 80 is further provided in the present disclosure, to make the sliding rod 1 and the supporting assembly 4000 form at least one buffering structure in the axial direction. In the present embodiment, the axial buffering mechanism 80 includes two buffering members, specifically being a first buffering member 8 and a second buffering member 11. The first buffering member 8 and the second buffering member 11 are arranged on both sides of the supporting assembly 4000. The two buffering members are springs. The first buffering member 8 is provided between the check ring 9 and the insulating member 10, and the second buffering member 11 is provided between the first end of the holding part 3 and the check rim 101 of the insulating member 10. Both the first buffering member 8 and the second buffering member 11 are sleeved on the sliding rod 1, and are provided in a pre-compression state in the insulating member 10. The first buffering member 8 and the second buffering member 11 produce buffering when the sliding rod 1 slides with respect to the supporting assembly 4000. When the sliding rod 1 slides, a portion of the impact on the supporting assembly 4000 is absorbed by the buffering members. Thus, when the sliding rod 1 slides axially to install the prosthesis 1003, the sliding rod 1 will not provide rigid impact on the robotic arm 9100, thereby reducing locking state or a posture error of the robotic arm 9100.

With driving by the robotic arm 9100, the second actuator 7000 achieves a target aligning posture for installing the acetabular prosthesis, and the prosthesis 1003 is aligned with the prepared acetabular fossa at the affected area of a patient. During moving and positioning of the robotic arm 9100, both the first buffering member 8 and the second buffering member 11 are in a compression state. Under the effect of the first buffering member 8 and the second buffering member t 11, the sliding rod 1 maintains a certain axial positioning relation with respect to the supporting member 4. That is, the sliding rod 1 is maintained about in a middle position of the sliding stroke, and will not move freely along the accommodating channel 5.

After the surgeon confirms that the posture of the prosthesis 1003 and the surgical path are correct, the robotic arm 9100 is configured to a mode of linear spring arm. That is, by controlling output torque of a motor at a joint of the robotic arm 9100, the robotic arm 9100 is configured such that the damping on its end arm/rod is very low in the axial direction of the sliding rod 1, but is very high in other directions. In this mode, the second actuator 7000 connected to the robotic arm 9100 may, under the action of the external force, move in the axial direction of the sliding rod 1, but it is difficult for it to move radially or rotate around an axis in the radial direction. The surgeon holds the holding part 3 in hand and applies the impact on the first end of the sliding rod 1. The impact may be applied by hitting with a hammer or a sliding weight. With the impact, the sliding rod 1 drives the prosthesis 1003 to enter into the acetabulum. At a moment of the impact, the supporting assembly 4000 will not move at once due to the effect of inertia. During movement of the sliding rod 1, the check ring 9 compresses the first buffering member 8, and the first buffering member 8 acts on the supporting assembly 4000, to make the supporting assembly 4000 moves axially with the sliding rod 1 in a delayed manner. The first buffering member 8 prevents a rigid contact between the spring check ring 9 and the supporting member 4. After the sliding rod 1 completes one impact on the prosthesis 1003, under the effect of the first buffering member 8, the relative relation between the sliding rod 1 and the supporting assembly 4000 automatically restores to the state before hammering. In some cases, it is further necessary to apply a force in a direction opposite to the direction of the hammering force during implanting the prosthesis to the second actuator 7000, to remove the prosthesis 1003 or a prosthesis testing sample from the acetabulum. In this case, the second buffering member 11 may prevent a rigid contact between the sliding rod 1 and the supporting assembly 4000. With configuration of the above-mentioned buffering mechanism, during impacting the sliding rod 1, the robotic arm 9100 may automatically move with the sliding rod 1, without manually holding the actuator. An operator may hold the sliding rod 1, and may sense the impact and shock, like in traditional surgeries.

A stroke of axial moving of the sliding rod 1 is defined by the first end of the holding part 3 and the check ring 9, of the limit structure. The first buffering member 8 and the second buffering member 11 are provided such that the limit structure of the sliding rod 1 is never in rigid contact with the supporting member 4. When the sliding rod 1 is not receiving the impact, the sliding rod 1 is maintained in a middle position with respect to the accommodating channel 5, and the sliding rod 1 will not freely move with respect to the supporting assembly 4000. Rather, a certain force is necessary to overcome the effect of the first buffering member 8 or the second buffering member 11 to make the sliding rod 1 move, and thus avoiding any random movement of the sliding rod 1 during movement of the robotic arm 9100.

In an optional embodiment, the buffering member may include only the first buffering member 8, and omitting the second buffering member 11.

In some optional embodiments, it is possible to provide one buffering member, such as the buffering member 8. Two ends of the buffering member 8 are connected to the check ring 9 and the supporting assembly 4000, respectively. Therefore, the sliding rod 1 will be pulled or stopped by the buffering member 8 when it moves in either of the two directions, and thus forming buffering and possibly driving the supporting assembly 4000 to move with the sliding rod 1.

In some optional embodiments, the two buffering members of the axial buffering mechanism 80 may not be pre-compressed. For example, the first buffering member 8 may be compressed only by effect of gravity of the sliding rod 1. Lengths of the two buffering members may be less than the stroke of the sliding rod 1, and the buffering members may move between the limit structures as long as any rigid collision is capable to be avoided.

In an optional embodiment, the supporting assembly 4000 is provided thereon with a quick release mechanism 140 for connecting the second actuator 7000 with the robotic arm 9100 or the first actuator 6000. As shown in FIGS. 23 to 25, the quick release mechanism 140 includes a first limit mechanism 141 and a second limit mechanism 142. The first limit mechanism 141 is an inserting block 12, and the second limit mechanism 142 is a locking pin assembly. The inserting block 12 is used for connecting with the robotic arm 9100 or the first actuator 6000 in an insert-connection manner. An insert-connection limit direction of the locking pin assembly is perpendicular to the inserting-joint direction of the inserting block 12. The inserting block 12 and the supporting member 4 are fixedly connected or integrally formed. The insert block 12 is provided, on an end thereof along its inserting-joint direction, with two limit grooves 121 for limiting degree of freedom in the inserting-joint direction.

The supporting member 4 is provided thereon with an installing hole 14 for accommodating the locking pin assembly, and the installing hole 14 communicates with the accommodating channel 5. The locking pin assembly includes a locking pin 15, a first elastic member 16, a spacer block 17 and a locking pin pulling bolt 18. The spacer block 17, the first elastic member 16 and the locking pin 15 are provided in sequence in the installing hole 14. The first elastic member 16 is a spring. The spacer block 17 abuts against the sliding rod 1. The locking pin 15 is in the installing hole 14 and penetrates through the inserting block 12 perpendicularly in a thickness direction of the inserting block 12. The first elastic member 16 is in a compressed state, and is provided between the locking pin 15 and the spacer block 17. A middle section of the installing hole 14 communicates with an outside of the supporting member 4, and forming a movable region in which the locking pin 15 is capable to be manually operated. The locking pin pulling bolt 18 radially penetrates through the locking pin 15, and is fixed with the locking pin 15. The locking pin 15 is restricted in the movable region by the locking pin pulling bolt 18. With pushing by the first elastic member 16, the locking pin pulling bolt 18 abuts against an end of the movable region, and a head of the locking pin 15 penetrates through a surface of the inserting block 12. The head of the locking pin 15 is an inclined face.

In order to install the second actuator 7000 to the first actuator 6000 by the quick release mechanism 140, the first actuator 6000 is provided thereon with a second connector 13 in an inserting slot form. Specifically, the second connector 13 includes a bottom plate 131, a locking pin hole 133 and a limit button 132, where the bottom plate 131 is in a rectangular shape. The locking pin hole 133 is provided in a thickness direction of the bottom plate 131. The number of the limit buttons 132 is four, and they are provided at four corners of the bottom plate 131, respectively. The limit buttons 132 and the bottom plate 131 form the second connector 13. The limit button 132 specifically includes a first section 1321 and a second section 1322 which are connected to each other. The first section 1321 is connected to the bottom plate 131, and is perpendicular to the bottom plate 131. The second section 1322 is parallel to the bottom plate 131, and extends towards an inside of the bottom plate 131. The limit buttons 132 and the bottom plate 131 form a space accommodating the inserting block 12. Moreover, when the inserting block 12 is inserted and connected into the second connector 13, the limit groove 121 is in clamping-connection with the limit button 132, and with the limitation by the limit buttons 132, the inserting block 12 cannot be disengaged from the clamping groove in the insert-connection direction.

By configuration of the quick release mechanism 140, it may be convenient to assemble/disassemble the second actuator 7000. As shown in FIGS. 23 to 25, when the inserting block 12 is connected to the second connector 13 from top to bottom, the plane of the bottom plate 131 is first fitted the plane of the inserting block 12, the inclined face of the head of the locking pin contacts with the bottom plate 131, and the locking pin 15 retracts towards the supporting member 4. The supporting member 4 is moved downwards with respect to the second connector, the limit groove 121 and limit button 132 are in clamping-fitted, the head of the locking pin 15 enters into the locking pin hole 133, and the inserting block 12 and the second connector 13 are completely fitted. In the spatial rectangular coordinate system, the fitness in thickness and width between the inserting block 12 and the second connector 13 defines five degrees of freedom of the inserting block 12, except a z axis (or may be an x axis or a y axis), the clamping-fitting between the limit groove 121 and the limit button 132 defines a degree of freedom of the second actuator 7000 sliding along the z axis in a first direction, and the fitting between the locking pin 15 and the locking pin hole 133 enables a degree of freedom of the second actuator 7000 sliding along the z axis in a second direction. In FIGS. 23 to 25, the first direction is a downward direction along the axial direction of the accommodating channel 5, and the second direction is an upward direction along the accommodating channel 5. By now, the second actuator 7000 is fixedly connected to the first actuator 6000 by the configuration of the inserting block 12, the second connector 13 and the locking pin assembly. For disassembling, it is only necessary to move the locking pin pulling bolt 18 (moving to the left in FIG. 23) to remove the head of the locking pin 15 from the locking pin hole 133, and then pull out (pulling upwards with respect to the second connector 13 in FIG. 23) the inserting block 12 from the second connector 13. With the configuration of the quick release mechanism 140 for the second actuator 7000, a surgeon during surgery may complete installing and removing of the second actuator 7000 quickly, and thus saving surgery time.

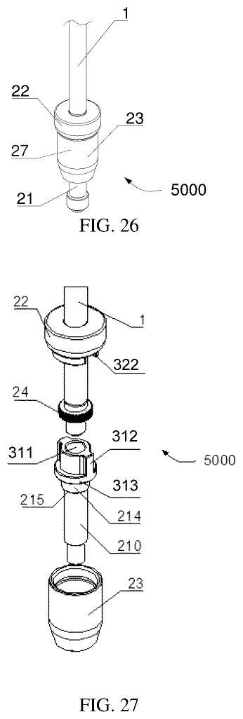

As shown in FIG. 26, in an optional embodiment, the second actuator 7000 further includes an adjusting assembly 5000. The adjusting assembly 5000 connects the prosthesis 1003 to the sliding rod 1, and is capable to adjust a circumferential position of the prosthesis 1003 with respect to the sliding rod 1. The adjusting assembly 5000 includes an adapter shaft 21 and an adjusting member 27. An end of the adapter shaft 21 is connected to the sliding rod 1, and the other end of the adapter shaft 21 is connected to the prosthesis 1003 of the hip joint. The adjusting member 27 is sleeved on a joint between the adapter shaft 21 and the sliding rod 1. With the effect of an external force, the adjusting member 27 may move between a first position 28 and a second position 29 of the adapter shaft 21. The adjusting member 27 is circumferentially fixed with the sliding rod 1 at the first position 28, and the adjusting member 27 is circumferentially adjustable with respect to the sliding rod 1 at the second position 29.

As shown in FIG. 27, the adapter shaft 21 includes a sliding rod connection joint, a main shaft section 210 and an acetabulum prosthesis connection joint. The sliding rod connection joint and the acetabulum prosthesis connection joint are provided at both ends of the main shaft section 210, respectively. The sliding rod connection joint is used for connecting with the sliding rod 1. The acetabulum prosthesis connection joint is used for connecting the prosthesis 1003.

The sliding rod connection joint is provided, at its top end, with a connecting hole 311. The connecting hole 311 is a smooth hole, and it is provided, on its outer periphery, with two clamping blocks 312 which are symmetrical with respect to the axis of the adapter shaft 21 and radially extends in a form of a Chinese character “—”. Below the clamping block 312, a flange 313 is provided, having a radius equal to the maximum radius of the clamping block 312. Below the flange 313, a limit section 214 is provided, having a radius larger than a radius of the main shaft section 210. Moreover, a limit step 215 is formed at a joint between the limit section 214 and the main shaft section 210.

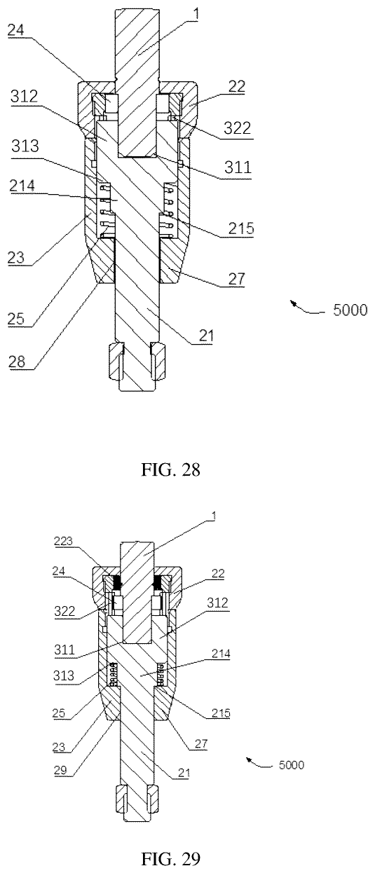

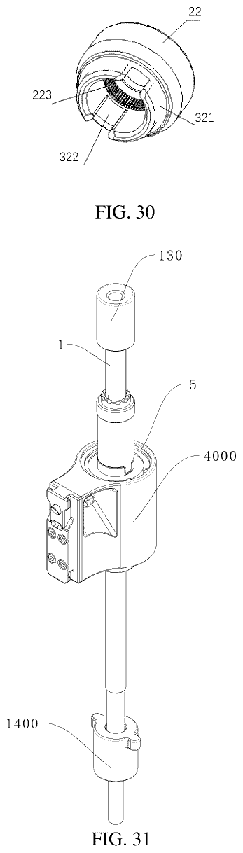

Referring to FIGS. 27 to 30, the adjusting member 27 includes a nut 22 and a reducing sleeve 23 detachably connected, a spline 24 and a holding member 25. Specifically, referring to FIG. 30, the nut 22 is in a shell shape with a downward opening, and includes an outer wall 321 which is provided thereon, at the opening, with an outer thread, and is also provided thereon with two clamping grooves 322 symmetrically. The clamping groove 322 extends into the nut 22. A spline keyway 223 is provided in a position in the nut 22 close to the bottom. The reducing sleeve 23 is in a cup shape with an opening, and is provided, on its inner wall at the opening, with an inner thread. The spline 24 is fixed on the sliding rod 1, and is provided on its outer periphery with a toothed bulge. The holding member 25 is a resilient spring.

In the connected state, the nut 22 is sleeved on the sliding rod 1 above the spline 24, the reducing sleeve 23 is sleeved on the adapter shaft 21, and the reducing sleeve 23 and the nut 22 are connected by fitting of the inner thread and the outer thread. The holding member 25 is provided in the reducing sleeve 23, with one end abutting against the bottom of the reducing sleeve 23 and the other end abutting against the flange 313.

In use, the end of the sliding rod 1 is inserted into the connecting hole 311, and the nut 22 and the reducing sleeve 23 form an integrated structure by thread connection. To facilitate understanding, the explanation will be provided hereinafter in combination with the operation state and the adjusting process of the adjusting member 27.

In the operation state, the adjusting member 27 is in the first position 28. As shown in FIG. 30, the holding member 25 is in the compressed state and abuts against with the flange 313 and the bottom of the reducing sleeve 23. The holding member 25 pulls the nut 22 through the reducing sleeve 23, to make the spline keyway 223 of the nut 22 is connected to the spline 24. The clamping block 312 is embedded in the clamping groove 322. Thus, the sliding rod 1 and the adjusting device are circumferentially fixed by connection of the spline 24 and the spline keyway 223, and the adapter shaft 21 and the adjusting apparatus are circumferentially fixed by cooperation of the clamping block 312 and the clamping groove 322. Based on the above-mentioned process and principle, in the operation state, by the connection of the adjusting assembly 4000, the sliding rod 1 and the adapter shaft 21 are fixed axially, radially and circumferentially.

In order to meet clinic requirements, when the prosthesis 1003 is implanted into the prepared acetabular fossa of an affected area of a patient, it is necessary to ensure that the prosthesis 1003 is in the correct installing direction. For example, for the prosthesis 1003 having a wing part, it is necessary for it to be fixed to the acetabular fossa to strengthen the structure at the acetabular fossa, and it is also necessary for the wing part to be connected to the acetabular fossa in the correct direction. Therefore, each time before the sliding rod 1 is moved, it is necessary to adjust the direction of the prosthesis 1003. Based on the second actuator 7000 in the present embodiment, when the direction of the prosthesis 1003 is adjusted, as shown in FIG. 31, a surgeon pulls the adjusting device upwards to make it overcome the elastic force of the holding member 25 until the bottom of the reducing sleeve 23 abuts against the limit step 215. The adjusting member 27 is located in the second position 29. At this time, the spline 24 is disengaged from the spline keyway 223, and the clamping block 312 is not removed from the clamping groove 322, the adjusting member 27 may circumferentially rotate with respect to the sliding rod 1, and the adapter shaft 21 rotates with rotation of the adjusting member 27. Thus, it is possible to achieve the adjustment the direction of the prosthesis 1003 with respect to the sliding rod 1 only by rotation of the adjusting member 27, without rotation of the sliding rod 1. Further, as the sliding rod 1 is connected to a tracer 2 for providing position information of the sliding rod 1 in real time, and it is necessary for the tracer 2 to be aligned with the positioner for receiving the position information. Therefore, the above-mentioned configuration of the adjusting assembly 5000 also ensures that when the prosthesis 1003 is adjusted, the tracer 2 fixedly connected to the sliding rod 1 will not fail in aligning with the positioner due to rotation of the sliding rod 1, and thus ensuring that the tracer 2 is capable to be identified by the positioner in real time.

Furthermore, based on the adjusting assembly 5000, by changing the acetabulum prosthesis connection joint of the adapter shaft 21, the adapter shaft 21 may connect with the prostheses 1003 of various models from different manufacturers. Therefore, it is not necessary to replace the whole sliding rod 1 to adapt to a different prosthesis 1003, and thus the adaptation and application range of the second actuator 7000 is improved.

With continued reference to FIG. 1, in a second aspect of the present disclosure, a surgical system is provided, including: the actuator for the hip replacement 9300 as provided in the first aspect, a robotic arm 9100, a navigation system 9000 and a controlling system 9200. The robotic arm 9100 is configured to carry the actuator for the hip replacement, the navigation system 9000 is used for measuring a position of the actuator for the hip replacement 9300, and the controlling system 9200 is configured to drive the robotic arm 9100 to move the actuator for the hip replacement 9300 to a target position according to a surgical plan.

The robotic arm 9100 may completely actively control the orientation of the actuator, and may also limit some degree(s) of freedom of movement or range of movement. of the actuator, in a cooperation manner. Specifically, by programming of the controlling system 9200, it is possible to control the robotic arm 9100 to make it completely independently move according to the surgical plan, or provide tactile feedback or force feedback to prevent the surgeon from manually moving beyond a predetermined virtual boundary, or provide virtual guidance to guide the surgeon to move in a certain degree of freedom. The virtual boundary and the virtual guidance may come from the surgical plan, or may be configured by an input device during surgery. The actuator is detachably connected to the robotic arm 9100.

The navigation system 9000 is used for measuring the positions of the actuator and a patient. The navigation system 9000 generally includes a positioner and a tracer. The tracers are installed on the actuator, a surgical tool and a body of a patient. Generally, the tracer is an array consisting of a plurality of tracing elements, and each tracing element may transmit an optical or electromagnetic signal or the like in an active or passive manner. The positioner (such as a binocular camera) measures the orientation of the above-mentioned tracer by 3D measuring technique.

The controlling system 9200, according to the position information obtained by the navigation system 9000, compares the current position and a target position of the actuator, and drives the robotic arm 9100 to move the actuator for the hip replacement 9300 to the target position according to the surgical plan. In the surgical plan, it is possible to include a moving path, a movement boundary, and the like of the robotic arm. The surgical plan is recorded in a three-dimensional (3D) reconstruction digital model for an affected bone of a patient, and the digital model will be registered with the tissue of the patient during surgery.

In the surgical system, with the assistance of the robotic arm 9100, the controlling system 9200 and the navigation system 9000, it is capable to perform preparation of the acetabular fossa or the medullary cavity at an affected area, with only the first actuator 6000 connected, and it is capable to perform installing of the prosthesis 1003, with the second actuator 7000 connected to the first actuator 6000.

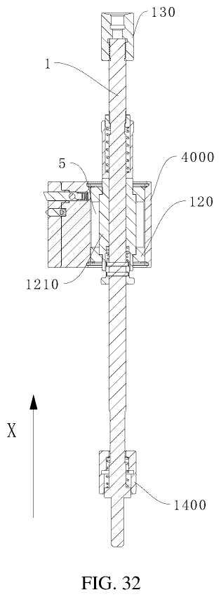

FIGS. 31 to 33 show structural schematic diagrams of the prosthesis installing actuator in another embodiment. In the present embodiment, the prosthesis installing actuator differs from the actuator as shown in FIGS. 18 to 20 mainly in that a first damping mechanism is added between the sliding rod 1 and the accommodating channel 5, and the first damping mechanism is capable to at least provide radial buffering. The prosthesis installing actuator may be used in combination with the above-described prosthesis forming actuator.

It is found in research by an inventor that during a hip replacement surgery with robot assistance, the surgery device includes: an acetabulum prosthesis (acetabular cup), a prosthesis installing actuator, a bone hammer and a robotic arm or the like. The prosthesis installing actuator includes a connecting mechanism for connecting the robotic arm and a sliding rod connected to the connecting mechanism. During surgery, it is necessary to use the hammering on the sliding rod 1 in the prosthesis installing actuator by the bone hammer to hammer the acetabulum prosthesis into the acetabular fossa of a patient. The shock (also called impact) during hammering is transmitted from the sliding rod 1 to the connecting mechanism, and then further to the robotic arm 9100. When the hammering force is high and the shock is violent, there will be a risk of locking fault for the robotic arm. The locking fault of the robotic arm will not only damage the robotic arm itself, but also decrease reliability of the robotic arm, and further will affect smooth of a surgery process and put the patient in danger. Based on the study on the above-mentioned problem, a prosthesis installing actuator is provided by the inventor, which may effectively solve the problem that the impact received by the prosthesis installing actuator is easily transmitted to the robotic arm, and thus the reliability of the robotic arm connected to the prosthesis installing actuator is improved.

In order to better understand the present disclosure, a prosthesis installing actuator according to the embodiment of the present disclosure will be described in details hereinafter in combination with FIGS. 31 to 33.

Please refer to FIGS. 31 to 33. In the embodiment of the present disclosure, a prosthesis installing actuator is provided, configured to perform a surgical plan with holding by the robotic arm 9100. This prosthesis installing actuator includes a sliding rod 1, a supporting assembly 4000 and a damping apparatus. The sliding rod 1 is used for carrying a prosthesis and is capable to receive the impact to install the prosthesis to a target position. The supporting assembly 4000 includes an accommodating channel 5. The accommodating channel 5 is used for accommodating the sliding rod 1, and is configured such that the sliding rod 1 is capable to move linearly with respect to the accommodating channel 5. A diameter of the accommodating channel 5 is larger than a diameter of the sliding rod 1.

In a possible embodiment, the damping apparatus is a first damping mechanism 120. The first damping mechanism 120 is provided in the accommodating channel 5, and the first damping mechanism 101 is configured to join the sliding rod 1 on a circumferential side, for reducing the impact transmitted from the sliding rod 1 to the supporting assembly 4000. The first damping mechanism 120 is capable to resiliently hold the sliding rod 1 in the accommodating channel 5, and is capable to make the sliding rod 1 and the accommodating channel 5 maintain substantially coaxial and do not contact with each other. When the sliding rod 101 is deflected or moved radially by a radial force, it will not directly collide rigidly with the supporting assembly 4000 due to elastic deformation of the first damping mechanism 120, and thus a buffering requirement and a requirement of positioning precision of the system are both met.

Where, the prosthesis is the acetabulum prosthesis, also called an acetabular cup. The supporting assembly 4000 is configured to connect with the robotic arm 9100, and the robotic arm 9100 holds the sliding rod 1 by connecting with the supporting assembly 4000. In the prosthesis installing actuator as provided in the present disclosure, the first damping mechanism 120 may achieve installation of the first damping mechanism 120 and the supporting assembly 4000 by interference fitting with the accommodating channel 5. The supporting assembly 4000 may be slidably connected to the sliding rod 1, and defines a sliding stroke of the sliding rod 1 in a length direction x of the sliding rod 1 by the limit mechanism.

In the prosthesis installing actuator as provided in the present disclosure, the accommodating channel 5 in the supporting assembly 4000 is used for accommodating the sliding rod 1 and the first damping mechanism 120. The sliding rod 1 penetrates through the accommodating channel 5, that is, the supporting assembly 4000 is sleeved on the sliding rod 1. The first damping mechanism 120 is provided between the sliding rod 1 and the accommodating channel 5. The first damping mechanism 120 is provided on a circumferential side of the sliding rod 1, it is possible to use the circumferential side of the sliding rod 1 to sufficiently reduce the impact transmitted from the sliding rod 1 to the supporting assembly 4000, therefore the impact influence on the robotic arm connected to the supporting assembly 4000 is effectively reduced, and thus probability of locking fault of the robotic arm connected with the prosthesis installing actuator due to the impact is decreased, and consequently, the reliability of the robotic arm is improved, and also probability of success of the surgery is increased.

In a possible embodiment, as shown in FIG. 31, the prosthesis installing actuator further includes a hitting cap 130 and an acetabular cup connecting mechanism 1400. The hitting cap 130 is provided on an end of the sliding rod 1, and the acetabular cup connecting mechanism 1400 is provided at the other end of the sliding rod 1. The acetabular cup connecting mechanism 1400 is used for connecting the prosthesis.

In the prosthesis installing actuator as provided in the present disclosure, the hitting cap 130 is connected to one end of the sliding rod 1, and the prosthesis connecting mechanism 14 is connected to the other end of the sliding rod 1. During use of the prosthesis installing actuator, that is, during surgery, the hitting cap 130 is the portion receiving the force of hammering. The sliding rod 1 and the hitting cap 130 are fixed to form an integrated structure. By hammering the hitting cap 130 by a bone hammer, the prosthesis connected to the acetabular cup connecting mechanism 1400 is installed into an acetabular fossa of a patient.

In a possible embodiment, the first damping mechanism 120 includes a first elastic member 122. The first elastic member 122 has ability of deformation in the radial direction and/or axial direction of the accommodating channel 5, and thus is capable to absorb the deformation in the radial direction and/or axial direction. When the sliding rod 1 receives the impact, the first elastic member 122 may radially and/or axially reduce transmission of the impact on the sliding rod 1 to the supporting assembly 4000, and thus protecting the robotic arm connected to the supporting assembly 4000.

In a possible embodiment, the first damping mechanism 120 further includes a sleeve 1210, with its inner circumference in shaft-hole fit with the sliding rod 1 and its outer circumference fitting with the first elastic member 122.

In the above-mentioned embodiment, the sleeve 1210 is sleeved on the circumferential side of the sliding rod 1, and the first elastic member 122 is provided on a circumferential side of the sleeve 1210, to achieve buffering between the sliding rod 1 and the supporting assembly 4000 comprehensively.

In a possible embodiment, the first elastic member 122 is a damping pad which is in a ring shape and has a predetermined axial thickness and radial thickness.

In the above-mentioned embodiment, the first elastic member 122 is the damping pad, and its material may be silica gel. The material of silica gel has a relatively good elasticity, and may provide good buffering and damping effect, with such material widely available and convenient for installing. The damping pad may deform in response to impact to achieve shock absorption, thus reducing transmission of the shock from the sliding rod 1 to the supporting assembly 4000. The damping pad may in a ring shape such that it is capable to be sleeved on the circumferential side of the sleeve 1210. The damping pad may be installed to the sleeve 1210 in an interference fitting manner, to improve connecting compactness and better absorb the impact transmitted from the sliding rod 1 to the sleeve 1210. The damping pad has a predetermined axial thickness and radial thickness, and thus may provide buffering in the axial and/or radial direction. It is possible to configure the axial thickness and the radial thickness of the damping pad according to the impact in a practical operation, and they are not specifically defined in the present disclosure.

In the above-mentioned embodiment, the damping pad is sleeved on the circumferential side of the sleeve 1210, and the sleeve 1210 is sleeved on the circumferential side of the sliding rod 1. Thus, the shock absorption function in the circumferential direction of the sliding rod 1 may be achieved. Moreover, as the damping pad achieves the shock absorption function by elastic deformation under pressure and it may deform under pressure in any direction, the shock absorption effect is capable be available in any direction except for the circumferential direction. That is, the first damping mechanism 120 may reduce the shock transmitted from the sliding rod 1 to the supporting assembly 4000 comprehensively in multiple directions.

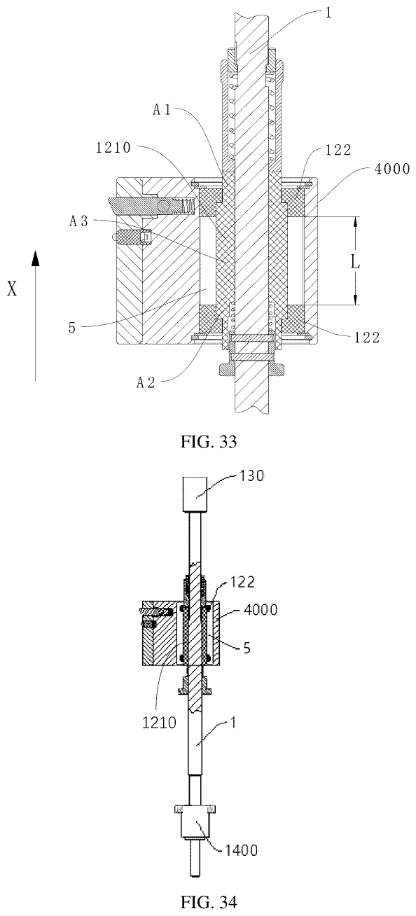

In a possible embodiment, as shown in FIG. 33, the sleeve 1210 includes a first end A1 and a second end A2 in the length direction x of the sliding rod 1. There are two damping pads, where one damping pad is sleeved at the first end A1 and the other damping pad is sleeved at the second end A2. In the length direction x of the sliding rod 1, a preset gap L is formed between the two damping pads.

In the above-mentioned embodiment, there are two damping pads which are arranged in the length direction x of the sliding rod 1. In the length direction x of the sliding rod 1, the preset gap L is formed between the two damping pads, such that the two damping pads are independent with respect to each other. When the shock is produced, the two damping pads may deform individually to achieve shock absorption, without interference with each other, such that each damping pad may deform more flexibly. The two damping pads may effectively reduce transmission of the shock, respectively, and thus the shock absorption effect of the first damping mechanism 120 is improved.

In the above-mentioned embodiment, the damping pad may be installed to the sleeve 1210 in an interference fitting manner, and the damping pad may be installed to the supporting assembly 4000 in the interference fitting manner. In this case, as the damping pad, the sleeve 1210 and the supporting assembly 4000 are tightly fitted, so that tightness therebetween may be improved, and thus the possibility of infiltration of liquid from the preset gap L is decreased and reliability is improved.

In another possible embodiment, the number of the damping pad may be one. The damping pad extends from the first end A1 to the second end A2 of the sleeve 1210. Compared with the configuration of two damping pads, the amount of the used material is increased, but the number of the parts is decreased, so that complexity of assembling is lowered, and also the shock absorption effect remains, reducing the impact transmitted from the sliding rod 1 to the supporting assembly 4000.

In an optional embodiment, the first elastic member 122 may be a group of springs arranged in a circumferential direction of the sleeve 1210, and a direction of deformation of the springs is a radial direction of the accommodating channel. The springs are mainly configured to provide radial shock absorption between the sliding rod 1 and the supporting assembly 4000, and thus protecting the robotic arm connected to the supporting assembly 4000. Specifically, as shown in FIG. 34, outside the sleeve 1210 in which the sliding rod 1 is sleeved, four springs are uniformly provided in the circumferential direction, and a stretching/contraction direction of the spring is perpendicular to the axial direction of the sliding rod 1. In some optional embodiments, the four circumferentially arranged springs belong to one group, and it is possible to provide multiple groups of springs which are arranged in the axial direction of the sleeve 1210.

In a possible embodiment, as shown in FIG. 33, the sleeve 1210 includes a middle region A3 located between the first end A1 and the second end A2. Diameters of the first end A1 and the second end A2 are less than a diameter of the middle region A3 to form grooves, and the damping pad partially extends into the groove.

In the above-mentioned embodiment, the diameters of the first end A1 and the second end A2 of the sleeve 1210 refer to diameters of circumcircles of the first end A1 and the second end A2 of the sleeve 1210 along their own circumferential sides, and the diameter of the middle region A3 refers to a diameter of a circumcircle of the middle region A3 along its own circumferential side.

In the above-mentioned embodiment, by configuring the diameters of the first end A1 and the second end A2 of the sleeve 1210 as being less than the diameter of the middle region A3, the grooves may thus be formed at the first end A1 and the second end A2, respectively. That is, stepped surfaces are formed between the first end A1 and the middle region A3 and between the second end A2 and the middle region A3. This stepped surface is an inner surface of the groove. By configuring a cross-section in the damping pad, parallel to the length direction x of the sliding rod 1, as an “L” shape, such that one end of the L shape contacts with the stepped surface and the other end of the L shape contacts with an outer surface of the middle region A3, limit to the damping pad in the length direction x of the sliding rod 1 is achieved, and thus ensuring the two damping pads achieve the shock absorption effect at two ends of the sleeve 1210.

In existing designs, the supporting assembly and the robotic arm are rigidly connected together, such that the robotic arm is prone to be damaged or locking due to shock of the supporting assembly 4000. The shock of the supporting assembly 4000 mainly comes from the shock transmitted from the sliding rod 1 when it is hitting. In the prosthesis installing actuator in the present disclosure, by providing the first damping mechanism 120 and the axial buffering mechanism 80 between the supporting assembly 4000 and the sliding rod 1, the shock transmitted to the supporting assembly 4000 may be effectively reduced, so that effectively protecting the robotic arm, and thus reliability of the robotic arm is improved, ensuring that the surgery is performed smoothly, and thus loss in life and property is reduced.

Though the present disclosure has been described in detail as above with the general description and specific embodiments, it is possible to make some modification or improvement thereto based on the present disclosure, which will be obvious to a person skilled in the art. Therefore, such modification or improvement made based thereon, without departing from the spirit of the present disclosure, will fall within the protection scope of the present disclosure.

What is claimed is:

1. An actuator for hip replacement, which is configured to prepare a space for installing a prosthesis on a bone and implant the prosthesis, comprising:
a first actuator, configured to connect a cutting tool to process at least one of an acetabulum or a medullary cavity, the first actuator having a first connector and a second connector; and
a second actuator, configured to connect to the second connector of the first actuator during an operation of implantation of the prosthesis is performed, and connect the prosthesis and receive impact produced during installing the prosthesis,
wherein the actuator for the hip replacement is configured to be installed to a robotic arm via the first connector.

2. The actuator for the hip replacement according to claim 1, wherein when the second actuator is connected to the first actuator, a structure to be connected to the prosthesis is parallel to a structure to be connected to the cutting tool.

3. The actuator for the hip replacement according to claim 1, wherein the first actuator comprises a power apparatus and a tool assembly which are detachably connected to each other.

4. The actuator for the hip replacement according to claim 3, wherein the power apparatus comprises a built-in power assembly, the power assembly comprises a power source and an output shaft, and the output shaft is connected to the power source;
the tool assembly comprises a connecting part and a surgical tool, the surgical tool is rotatably provided on the connecting part, and the tool assembly is detachably provided on the power apparatus by the connecting part; and
when the tool assembly is connected to the power apparatus through the connecting part, the surgical tool is joined to the output shaft to receive rotation movement output by the output shaft.

5. The actuator for the hip replacement according to claim 4, wherein a radial positioning structure is further provided between the surgical tool and the power apparatus.

6. The actuator for the hip replacement according to claim 4, wherein the connecting part is connected with the power apparatus through a rotating joint structure, to form an axial and circumferential limit to the connecting part.

7. The actuator for the hip replacement according to claim 6, wherein the rotating joint structure comprises a spiral groove provided on a circumferential face and a positioning pin, and the spiral groove is used for guiding the positioning pin and comprises a limiting part for limiting an axial direction and a circumferential direction of the positioning pin.

8. The actuator for the hip replacement according to claim 4, wherein a positioning module is provided between the connecting part and the power apparatus, to form a predetermined acting force between the connecting part and the power apparatus.

9. The actuator for the hip replacement according to claim 1, wherein the second actuator is a prosthesis installing actuator, which comprises:
a sliding rod, wherein a first end of the sliding rod is configured to connect the prosthesis, and a second end of the sliding rod is configured to receive the impact produced during installing the prosthesis;
a supporting assembly, comprising an accommodating channel, wherein the accommodating channel accommodates a portion of a rod section of the sliding rod, and the sliding rod is axially movable with respect to the supporting assembly; and the supporting assembly is used for connecting the prosthesis installing actuator to the second connector of the first actuator; and
a tracer, which is provided on the sliding rod to indicate an orientation of the sliding rod.

10. The actuator for the hip replacement according to claim 9, further comprising an axial buffering mechanism, wherein when the sliding rod is subjected to axial impact, the axial buffering mechanism forms axial buffer between the sliding rod and the supporting assembly.

11. The actuator for the hip replacement according to claim 10, wherein the accommodating channel is a channel penetrating through the supporting assembly, and the axial buffering mechanism comprises two buffering members which are provided on two ends of the channel, respectively.

12. The actuator for the hip replacement according to claim 9, wherein a quick release mechanism is provided between the supporting assembly and at least one of the robotic arm and the first actuator, and the prosthesis installing actuator is connected to one of the robotic arm and the first actuator by the quick release mechanism.

13. The actuator for the hip replacement according to claim 12, wherein the quick release mechanism comprises a first limit mechanism and a second limit mechanism, and the second limit mechanism is a mechanism for manually removing a limit.

14. The actuator for the hip replacement according to claim 9, further comprising an adjusting assembly for adjusting a circumferential position of the prosthesis with respect to the sliding rod, wherein the adjusting assembly comprises:
an adapter shaft, an end of the adapter shaft being used for connecting with the prosthesis; and
an adjusting member, which is used for connecting the adapter shaft to the sliding rod, wherein a circumferential position of the adjusting member is adjustable with respect to the sliding rod, and a circumferential position of the adjusting member with respect to the adapter shaft is fixed.

15. The actuator for the hip replacement according to claim 9, further comprising a damping apparatus provided between the supporting assembly and the sliding rod, for reducing impact transmitted from the sliding rod to the supporting assembly.

16. The actuator for the hip replacement according to claim 15, wherein the damping apparatus is a first damping mechanism; an inner diameter of the accommodating channel of the supporting assembly is larger than a diameter of the sliding rod; the first damping mechanism is provided on the supporting assembly and is connected to the sliding rod, the first damping mechanism is constructed to enable the sliding rod to be elastically held in the accommodating channel, and enable a predetermined gap to be formed between the sliding rod and the accommodating channel.

17. The actuator for the hip replacement according to claim 16, wherein the first damping mechanism has at least ability of radial elastic deformation, to allow the sliding rod to deflect or move radially in the accommodating channel.

18. The actuator for the hip replacement according to claim 16, wherein the first damping mechanism comprises a first elastic member having ability to deform along a radial direction and/or an axial direction of the accommodating channel.

19. The actuator for hip replacement according to claim 18, wherein the first damping mechanism further comprises a sleeve, an inner periphery of the sleeve is in shaft-hole fit with a shaft hole of the sliding rod, and an outer periphery of the sleeve is fitted with the first elastic member.

20. A surgical system, comprising:
an actuator which is the actuator for the hip replacement according to claim 1;
a robotic arm, connected to the first connector of the actuator,
a navigation system, configured to measure a position of the actuator, and
a controlling system, configured to drive the robotic arm to move the actuator to a target position according to a surgical plan.
